(19) United States
(12) Patent Application Publication
(10) Pub. No.: US 2022/0257172 A1
(43) Pub. Date: Aug. 18, 2022

(54) DEMENTIA TEST DEVICE AND DEMENTIA DIAGNOSIS DEVICE, AND DEMENTIA DIAGNOSIS SYSTEM COMPRISING SAME

(71) Applicant: N.CER CO., LTD, Gwangju (KR)

(72) Inventors: Jae Won KIM, Gwangju (KR); Jae Gwan KIM, Gwangju (KR)

(21) Appl. No.: 17/610,742

(22) PCT Filed: Sep. 4, 2020
(86) PCT No.: PCT/KR2020/011939

§ 371 Date: Nov. 12, 2021

(30) Foreign Application Priority Data
Sep. 27, 2019 (KR) 10-2019-0120144

(51) Int. Cl. A61B 5/00; A61B 5/1455; G16H 50/30

(52) U.S. Cl. CPC A61B 5/4088; A61B 5/1455; A61B 5/4011; A61B 5/7275; A61B 5/0059; G16H 50/30; A61B 5/6814

(57) ABSTRACT

Disclosed are a dementia test device and dementia diagnosis device, and a dementia diagnosis system comprising same. Provided according to one aspect of the present embodiment are a dementia test device, dementia diagnosis device, and dementia diagnosis system which can accurately diagnose the possibility of dementia in a patient by analyzing the concentration of hemoglobin.

### TECHNICAL FIELD

The present invention relates to devices capable of objectively examining and diagnosing the possibility of dementia in a patient suspected of dementia and a diagnosis system including the same.

### BACKGROUND ART

The description of the Background Art section merely provides information that may be relevant to embodiments of the disclosure but should not be appreciated as necessarily constituting the prior art.

The conventional dementia diagnosis process proceeded as follows.

First, the examiner identifies whether there is a possibility of dementia among the patients by checking dementia-related symptoms, selects suspected patients, and determines whether the patient has the possibility of dementia using a questionnaire, such as the MMSE (Mini Mental Status Examination).

The examiner may identify the medical history of the suspected patient and carries out clinical evaluation, for specifically identifying dementia, such as, e.g., physical examination, blood test, daily life function evaluation, and cognitive function evaluation. Thereafter, the examiner determines dementia based on specific evidence, such as MRI or PET brain images of patients suspected of having dementia.

In this case, the MMSE, which is used to identify whether a patient has a possibility of dementia in the conventional dementia diagnosis process, is a test sheet for evaluating the patient's cognitive functions and has various questions for the cognitive function test. Based on whether the patient is able to answer the various questions listed in the MMSE, the examiner determines the patient's likelihood of dementia.

However, a test sheet, such as the conventional MMSE, gives in a certain degree of accuracy for patients who correctly understand the questions in the test sheet but presents inaccurate results for patients who do not understand the questions. In other words, a patient who does not have dementia and may answer the questions but does not understand the questions in the MMSE and thus gives incorrect answers, may be classified as a patient with the possibility of dementia according to the results of the MMSE test. Thus, an unnecessary additional test needs to be conducted to determine the possibility of dementia for such patients, causing inconvenience.

### DETAILED DESCRIPTION OF THE INVENTION

### Technical Problems

An embodiment of the present invention aims to provide a dementia test device, a dementia diagnosis device, and a dementia diagnosis system that objectively and accurately determine the possibility of dementia by determining the olfactory function activity or the tissue activity of the prefrontal lobe.

An embodiment of the present invention aims to provide a dementia test device, a dementia diagnosis device, and a dementia diagnosis system that provide a preset scent to all patients at an accurate distance to determine tissue activity.

An embodiment of the present invention aims to provide a dementia test device, a dementia diagnosis device, and a dementia diagnosis system capable of accurately diagnosing the possibility of dementia for a patient through machine learning.

An embodiment of the present invention aims to provide a dementia test device, a dementia diagnosis device, and a dementia diagnosis system capable of accurately diagnosing the possibility of dementia for a patient by analyzing the concentration of hemoglobin.

### Technical Solutions

According to an aspect of the present embodiment, there is provided a dementia test device attached to a preset portion of a patient to determine the olfactory function activity or the activity of prefrontal lobe, the dementia test device comprising a light source irradiating light of a preset wavelength band to the patient's prefrontal lobe and a light receiving unit receiving light reflected from the patient's prefrontal lobe.

According to an aspect of the present embodiment, there is provided a dementia diagnosis system attached to a preset portion of a patient and determining the activity of olfactory function or the activity of prefrontal lobe, the dementia diagnosis system comprising a test container disposed a preset distance away from the patient's olfactory organ and providing a preset scent, a dementia test device irradiating light of a preset wavelength band to the patient's prefrontal lobe and receiving light reflected from the patient's prefrontal lobe, and a diagnosis device analyzing information for the reflected light received by the dementia test device to analyze the activity of the olfactory function or the activity of the prefrontal lobe.

According to an aspect of the present embodiment, there is provided a dementia test device attached to a preset portion of a patient and examining a possibility of dementia for the patient, the dementia test device comprising a light source disposed on the preset portion to irradiate light of a preset wavelength band to the patient's prefrontal lobe and a plurality of light receiving units receiving light reflected from the patient's prefrontal lobe.

According to an aspect of the present embodiment, the preset portion is the patient's glabellar.

According to an aspect of the present embodiment, the light receiving units are arranged to face each of the left brain and the right brain.

According to an aspect of the present embodiment, the light receiving units are disposed apart from the light source by a predetermined distance.

According to an aspect of the present embodiment, there is provided a dementia diagnosis system examining a possibility of dementia for a patient, the dementia diagnosis system comprising a test container providing a scent to the patient's olfactory organ, a dementia test device irradiating light of a preset wavelength band to the patient's prefrontal lobe and receiving light reflected from the patient's prefrontal lobe, and a dementia diagnosis device analyzing information for the reflected light received by the dementia test device to derive the activity of the olfactory function or the prefrontal lobe and determine the possibility of dementia based on the activity derived by performing machine learning.

According to an aspect of the present embodiment, the dementia diagnosis device stores activity values or activity variations since a time when the scent is provided to the patient's olfactory organ according to whether each patient has dementia, as bigdata.

According to an aspect of the present embodiment, the dementia diagnosis device varies the light receiving sensitivity of the dementia test device while the dementia test device receives the reflected light.

According to an aspect of the present embodiment, there is provided a dementia diagnosis device examining a possibility of dementia for a dementia suspected patient by analyzing an amount of light reflected from the dementia suspected patient's prefrontal lobe in a situation where a scent for examination is provided to the dementia suspected patient, the dementia diagnosis device comprising an analyzer deriving a concentration of hemoglobin moving or having moved to the patient's prefrontal lobe before the scent is provided to the patient's olfactory organ and a concentration of hemoglobin moving or having moved to the patient's prefrontal lobe after the scent is provided to the patient's olfactory organ from the amount of the reflected light and analyzing a dementia possibility index based on the derived concentrations to diagnose the possibility of dementia for the dementia suspected patient.

According to an aspect of the present embodiment, the analyzer derives each of a concentration of oxy hemoglobin (Oxy Hb) and a concentration of deoxy hemoglobin (Deoxy Hb) in deriving the concentrations of hemoglobin.

According to an aspect of the present embodiment, the concentration of hemoglobin is a relative value with respect to a concentration of hemoglobin measured at each time and a preset reference value.

According to an aspect of the present embodiment, the analyzer determines whether the concentrations of hemoglobin before and after the scent is provided are varied by a preset ratio or more from the dementia possibility index and diagnoses the possibility of dementia for the dementia suspected patient according to a result of the determination.

According to an aspect of the present embodiment, there is provided a method for examining, by a dementia diagnosis device, a possibility of dementia for a dementia suspected patient by analyzing an amount of light reflected from the dementia suspected patient's prefrontal lobe in a situation where a scent for examination is provided to the dementia suspected patient, the method comprising deriving a concentration of hemoglobin moving or having moved to the patient's prefrontal lobe before the scent is provided to the patient's olfactory organ and a concentration of hemoglobin moving or having moved to the patient's prefrontal lobe after the scent is provided to the patient's olfactory organ from the amount of the reflected light and analyzing a dementia possibility index based on the derived concentrations to diagnose the possibility of dementia for the dementia suspected patient.

According to an aspect of the present embodiment, the deriving derives each of a concentration of oxy hemoglobin (Oxy Hb) and a concentration of deoxy hemoglobin (Deoxy Hb) in deriving the concentrations of hemoglobin.

According to an aspect of the present embodiment, there is provided a dementia diagnosis system examining a possibility of dementia for a patient, the dementia diagnosis system comprising a test container providing a scent to the patient's olfactory organ, a dementia test device irradiating light of a preset wavelength band to the patient's prefrontal lobe and receiving light reflected from the patient's prefrontal lobe, and a dementia diagnosis device analyzing information for the reflected light received by the dementia test device to derive a concentration of hemoglobin moving or having moved to the patient's prefrontal lobe before the scent is provided to the patient's olfactory organ and a concentration of hemoglobin moving or having moved to the patient's prefrontal lobe after the scent is provided to the patient's olfactory organ from the amount of the reflected light and analyzing a dementia possibility index based on the derived concentrations to diagnose the possibility of dementia for the dementia suspected patient.

### Advantageous Effects

As described above, according to an aspect of the present embodiment, it is possible to objectively and accurately determine the possibility of dementia by determining the olfactory function activity or the tissue activity of the prefrontal lobe.

According to an aspect of the present embodiment, it is possible to secure objectivity of the examination results by providing a preset scent to all patients at a precise distance away.

According to an aspect of the present embodiment, it is possible to accurately diagnose the possibility of dementia for a patient using machine learning.

Further, according to an aspect of the present embodiment, it is possible to objectively and accurately diagnose the possibility of dementia for a patient by analyzing the concentration of hemoglobin.

### BRIEF DESCRIPTION OF THE DRAWINGS

FIG. 1 is a view illustrating a configuration of a dementia diagnosis system according to a first embodiment of the present invention;

FIG. 2 is a view illustrating a configuration of a test container according to embodiments of the present invention;

FIG. 3 is a view illustrating a cross section of a test container according to embodiments of the present invention;

FIG. 4 is a view illustrating a configuration of a dementia test device according to the first embodiment of the present invention;

FIG. 5 is a view illustrating a configuration of a dementia test device according to a second embodiment of the present invention;

FIG. 6 is a view illustrating a configuration of a dementia diagnosis device according to the first embodiment of the present invention;

FIG. 7 is a flowchart illustrating a method for diagnosing a possibility of dementia for a patient by a dementia diagnosis system according to the first embodiment of the present invention;

FIG. 8 is a view illustrating a configuration of a dementia diagnosis system according to a third embodiment of the present invention;

FIG. 9 is a view illustrating a configuration of a dementia test device according to the third embodiment of the present invention;

FIG. 10 is a view illustrating a configuration of a dementia diagnosis device according to the third embodiment of the present invention;

FIG. 11 is a graph illustrating the hemoglobin concentration of a patient analyzed by a dementia diagnosis device according to the third embodiment of the present invention; and

FIG. 12 is a flowchart illustrating a method for diagnosing a possibility of dementia for a patient by a dementia diagnosis system according to the third embodiment of the present invention.

### MODE TO PRACTICE THE INVENTION

Various changes may be made to the present invention, and the present invention may come with a diversity of embodiments. Some embodiments of the present invention are shown and described in connection with the drawings. However, it should be appreciated that the present disclosure is not limited to the embodiments, and all changes and/or equivalents or replacements thereto also belong to the scope of the present disclosure. Similar reference denotations are used to refer to similar elements throughout the drawings.

The terms “first” and “second” may be used to describe various components, but the components should not be limited by the terms. The terms are used to distinguish one component from another. For example, a first component may be denoted a second component, and vice versa without departing from the scope of the present disclosure. The term “and/or” may denote a combination(s) of a plurality of related items as listed or any of the items.

It will be understood that when an element or layer is referred to as being “on,” “connected to,” “coupled to,” or “adjacent to” another element or layer, it can be directly on, connected, coupled, or adjacent to the other element or layer, or intervening elements or layers may be present. In contrast, when a component is “directly connected to” or “directly coupled to” another component, no other intervening components may intervene therebetween.

The terms as used herein are provided merely to describe some embodiments thereof, but not to limit the present disclosure. As used herein, the singular forms “a,” “an,” and “the” are intended to include the plural forms as well, unless the context clearly indicates otherwise. As used herein, the term “comprise,” “include,” or “have” should be appreciated not to preclude the presence or addability of features, numbers, steps, operations, components, parts, or combinations thereof as set forth herein.

Unless otherwise defined, all terms including technical and scientific terms used herein have the same meaning as commonly understood by one of ordinary skill in the art to which the embodiments of the present disclosure belong.

It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The components, processes, steps, or methods according to embodiments of the disclosure may be shared as long as they do not technically conflict with each other.

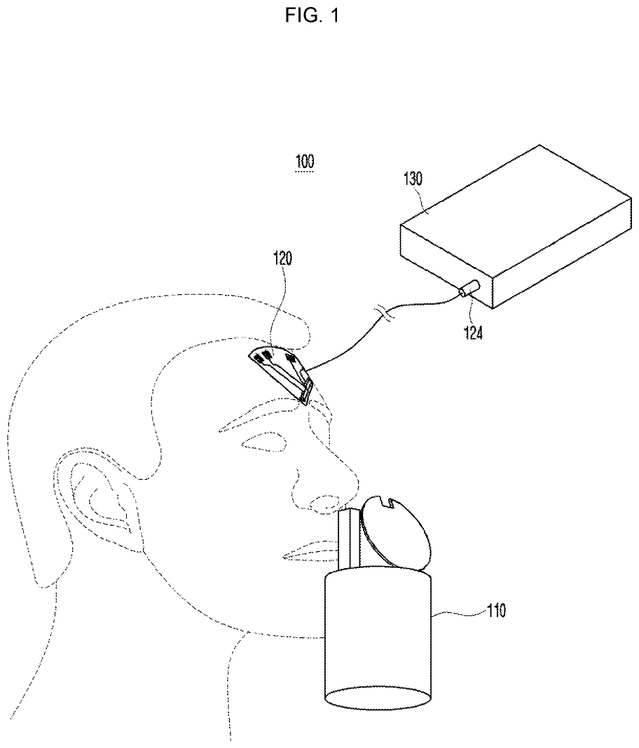

FIG. 1 is a view illustrating a configuration of a dementia diagnosis system according to a first embodiment of the present invention.

Referring to FIG. 1, the dementia diagnosis system 100 according to the first embodiment of the present invention includes a test container 110, a dementia test device 120, and a dementia diagnosis device 130.

Typically, if a human smells with the olfactory organ, the olfactory cells sense the smell and generate (biological) signals according to the smell. The prefrontal lobe receives the signals generated by the olfactory cells, regenerates signals, by which the part of the brain controlling the olfactory sense may process the signals, and transfers them to the part. Through the above-described process, humans perceive smells with their olfactory organs. In this case, the function of the prefrontal lobe begins to degenerate little by little as it ages, and such degeneration is more noticeable for dementia patients. Due to degeneration, although the same olfactory stimulus as that applied to non-dementia patients is applied to dementia patients, the activity of the prefrontal lobe decreases or, for processing of the same olfactory stimulus, it may be excessively activated. The dementia diagnosis system 100 detects this and diagnoses the possibility of dementia for a patient suspected of dementia.

The test container 110 provides a preset scent, a predetermined distant away from the patient's olfactory organ.

As described above, to determine the possibility of dementia, the activity of the olfactory function (activity of the prefrontal lobe function) should be determined. The test container 110 provides a preset scent to activate the olfactory organ of the dementia suspected patient so that the dementia test device 120 may test the olfactory function activity or the tissue activity of the prefrontal lobe of the dementia suspected patient.

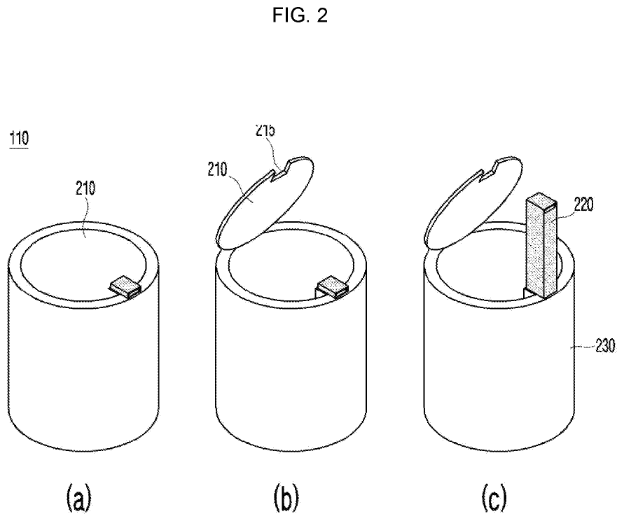

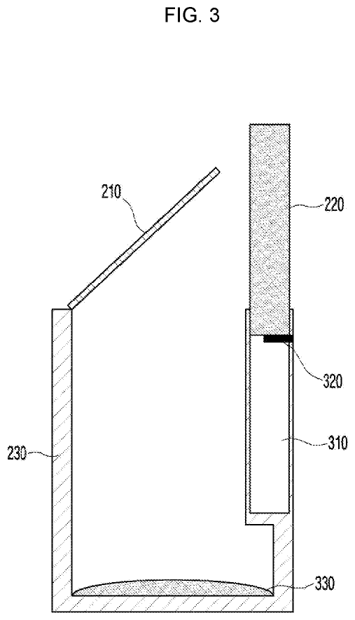

However, if the test container 110 provides a scent from a relatively short distance for a specific dementia suspected patient and from a relatively long distance for another dementia suspected patient, the accuracy of results of diagnosis by the dementia diagnosis system 100 may be reduced. To address this problem, the test container 110 has a structure that is physically separated from the patient's olfactory organ by a preset distance, and provides scent while being positioned the same distance apart for all patients. A detailed description of the structure of the test container 110 is described below with reference to FIGS. 2 and 3.

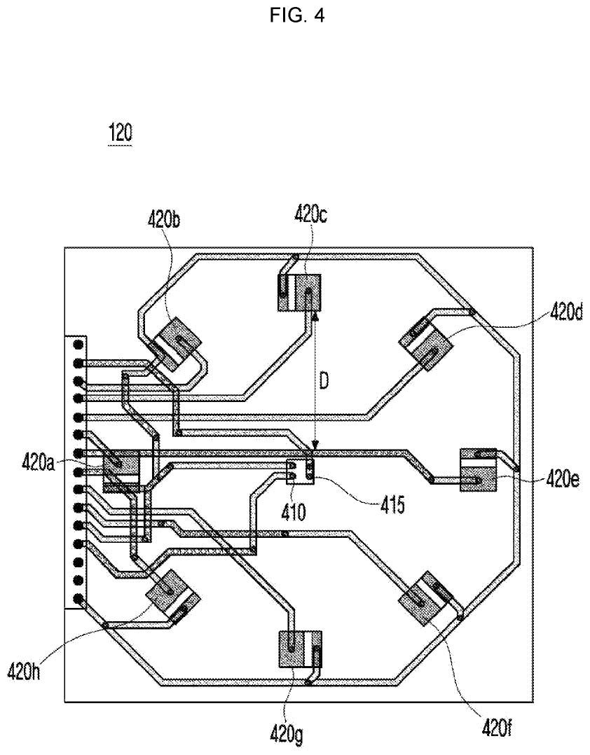

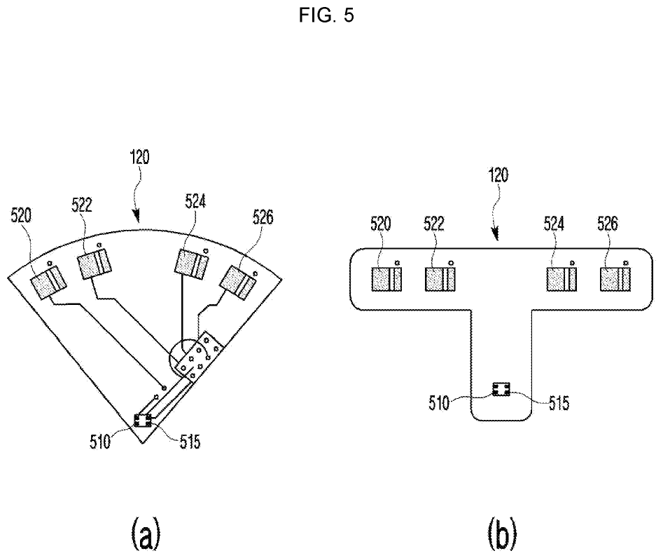

The dementia test device 120 may be attached to a preset portion of a dementia suspected patient and examines olfactory function activity or tissue activity of the prefrontal lobe. Since the test container 110 is provided to provide a scent to the dementia suspected patient, the olfactory function or the function of the prefrontal lobe of the dementia suspected patient is in an activated state. The dementia test device 120 is attached to a preset portion of the dementia suspected patient and examines the degree of tissue activity of the olfactory sense or prefrontal lobe (hereinafter, simply referred to as ‘tissue activity’) of the dementia suspected patient, activated by the test container 110. To examine the intensive degeneration of the prefrontal lobe function or the degeneration of the olfactory function shown from dementia patients, the dementia test device 120 is attached near the forehead of the dementia suspected patient. Since it may smoothly test tissue activity even with a small size when it is attached to an area close to the prefrontal lobe, the dementia test device 120 is attached to the vicinity of the forehead of the dementia suspected patient. The dementia test device 120 is attached to the forehead of the dementia suspected patient and then examines the tissue activity of the dementia suspected patient. The dementia test device 120 is described below with reference to FIG. 4 or 5.

The dementia test device 120 includes a connector 124, connected with the dementia diagnosis device 130, at one end thereof. The connector 124 is attached or detached to/from the dementia diagnosis device 130, thereby determining whether to electrically connect the dementia diagnosis device 120 and the dementia diagnosis device 130. Since the dementia test device 120 is attached to the dementia suspected patient in use, it needs to be replaced periodically or every time it is used. Accordingly, to facilitate replacement of the dementia test device 120 alone, the dementia test device 120 includes the connector 124 that is detachably coupled to the dementia diagnosis device 130.

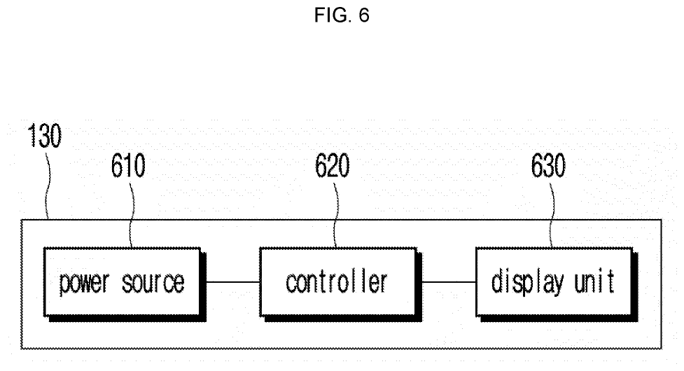

The dementia diagnosis device 130 analyzes the results of test by the dementia test device 120 to derive the tissue activity of the dementia suspected patient and analyzes the derived tissue activity by machine learning, thereby determining the possibility of dementia. The dementia diagnosis device 130 analyzes the results of test by the dementia test device 120 and derives the tissue activity of the patient as to how much the olfactory sense or prefrontal lobe of the dementia suspected patient has been activated by the scent provided from the test container 110. The dementia diagnosis device 130 matches the tissue activity of each of a large number of targets, to whether the target has dementia and stores them. The dementia diagnosis device 130 stores the tissue activities indicated by the test container and the possibility of dementia as big data and, based thereupon, performs machine learning and uses the tissue activity derived therefrom to diagnose the possibility of dementia for the patient. The dementia diagnosis device 130 is described below in greater detail with reference to FIG. 6.

FIG. 2 is a view illustrating a configuration of a test container according to an embodiment of the present invention. FIG. 3 is a view illustrating a cross section of a test container according to an embodiment of the present invention.

Referring to FIGS. 2 and 3, the test container 110 according to an embodiment of the present invention includes a cover 210, a separation unit 220, a case 230, a separation unit passage 310, a separation unit support 320, and a scent emissive material 330.

The cover 210 is formed on the uppermost end of the case 230, closing or opening the case 230 to control the transfer of the scent generated by the scent emissive material 330. The cover 210 minimizes the transfer of the scent generated by the scent emissive material 330 in the case 230 by covering and sealing the case 230 at the top of the case 230, or the cover 210 may be opened from the case 230 to allow the scent generated by the scent emissive material 330 in the case 230 to be transferred to the outside of the case 230.

To prevent the cover 210 from colliding with the separation unit 220 or the separation unit passage 310 when covering and sealing the case 230, the cover 210 includes a hole 215 in a preset area thereof where the separation unit 230 or the separation unit passage 310 is formed. To enhance the sealing effect of the cover 210 to cover and seal the case 230, the hole 215 formed in the portion of the cover 210 may have substantially the same area as the area of the separation unit 230 or the separation unit passage 310. Accordingly, if the cover 210 covers and seals the case 230, the hole 215 and the separation unit 230 or the separation unit passage 310 may air-tightly contact each other, preventing the scent from leaking to the outside of the case 230.

The separation unit 220 ascends and descends along the separation unit passage 310 and physically separates the case 230 from the olfactory organ of the dementia suspected patient by a preset distance. The separation unit 220 is positioned in the separation unit passage 310 if descending along the separation unit passage 310 and, if ascending along the separation passage 310, it protrudes from the case 230. One end of the separation unit 220 protruding from the case 230 may be fixed by the separation unit support 320, and the other end comes into contact with the olfactory organ of the dementia suspected patient. As two opposite ends of the separation unit 220 contact the separation unit support 320 and the olfactory organ, respectively, the separation unit 220 physically separates the case 230 and the olfactory organ by the length of the separation unit. Conventionally, in providing a scent, the examiner puts it an arbitrary distance apart from the olfactory organ of the dementia suspected patient by only relying on her own sense, causing inaccurate test results. By the separation unit 220, the test container 110 may provide a scent, the same distance away, whoever examiner proceeds with the test and whoever dementia suspected patient is tested, thus allowing for reliable tests.

The separation unit passage 310 is formed in a portion of the case 230 to allow the separation unit 220 to ascend or descend only in the passage without escaping off. The separation unit passage 310 has an area that is a predetermined extent larger than that of the separation unit 220 so that when the separation unit 220 is introduced into the separation unit passage 310, the separation unit may ascend or descend inly in the passage without escaping off the separation unit passage 310. The separation unit passage 310 supports the separation unit 220 at a certain height even when the separation unit 220 is fully lowered, allowing the examiner to easily raise or lower the separation unit 220 in use of the test container 110. Moreover, since the cover 210 includes the hole 215 through which the separation unit 230 or the separation unit passage 310 is exposed, the examiner may more easily raise or lower the separation unit 220.

The separation unit support 320 may be formed at a predetermined height of the separation unit passage 310, keeping the separation unit 220, which ascends and protrudes to the outside, at a fixed position. The separation unit 220 ascends and descends along the separation unit passage 310 and, when ascending to a preset height (protruding to the outside in a preset length), is supported by the separation unit support 320. The separation unit support 320 supports the raised separation unit 220, allowing the separation unit 220 to protrude by a preset height. Therefore, whoever dementia suspected patient is tested by the examiner using the test container 110, the separation unit support 320 allows the case 230 and the olfactory organ of the dementia suspected patient to be physically separated by a predetermined distance.

The scent emissive material 330 is disposed on the bottom of the case and emits a preset scent. The scent emissive material 330 generates any one scent of a (peanut) butter scent, peppermint scent, or gasoline smell, and preferably, three test containers 110 individually containing the scents may be used for dementia test for the dementia suspected patient.

FIG. 4 is a view illustrating a configuration of a dementia test device according to the first embodiment of the present invention.

Referring to FIG. 4, the dementia test device 120 includes light sources 410 and 415 and a light receiving unit 420.

The light sources are attached in the vicinity of the patient's forehead, in particular, between the patient's eyebrows and irradiate light into the patient's body (prefrontal lobe). In general, the prefrontal lobe, which controls the olfactory function, is located near the glabellar (the inside of the body, under the glabellar). Therefore, to accurately grasp the tissue activity of the patient, the light sources 410 and 415 are attached between the patient's eyebrows and irradiate light into the patient's body. The light sources 410 and 415 irradiate light in a near-infrared wavelength band.

Light in the near-infrared wavelength band has the property of being absorbed by hemoglobin. If the prefrontal lobe contains a lot of hemoglobin, the absorption of near-infrared light increases and the light reflection from the prefrontal lobe decreases. Hemoglobin is an oxygen carrier unless there is a special circumstance (such as carbon monoxide poisoning). Thus, an increase in the amount of hemoglobin means an increase in oxygen consumption in the tissue, thus meaning that the tissue is being activated. Accordingly, the light sources 410 and 415 irradiate near-infrared light into the patient's body to examine the olfactory function activity or the tissue activity of the prefrontal lobe of the dementia suspected patient.

The light sources 410 and 415 irradiate near-infrared light having different wavelengths. One of the light sources 410 and 415 irradiates light of a 730 nm band, and the other irradiates light of an 850 nm band. As such, as the near-infrared light of different wavelengths are irradiated from the light sources 410 and 415, it is possible to more accurately irradiate light to hemoglobin contained in the prefrontal lobe of the dementia suspected patient.

A plurality of light receiving units 420a to 420h are disposed apart from the light sources 410 and 415 by a predetermined distance, receiving the light reflected from the prefrontal lobe.

The light receiving units 420a to 420h receive the light reflected from the prefrontal lobe. The light receiving units 420a to 420h receive the light reflected, without being absorbed by hemoglobin, among the near-infrared light irradiated to the prefrontal lobe of the dementia suspected patient. As described above, the amount of reflected light varies depending on the amount of hemoglobin contained in the prefrontal lobe. Each of the light receiving units 420a to 420h transfers the sensed reflected light amount information to the diagnosis device 130 so that the diagnosis device 130 may determine the olfactory function activity or the tissue activity of the prefrontal lobe based on the amount of the reflected light.

The plurality of light receiving units 420a to 420h are disposed in different positions. If only one light-receiving part exists, obtainable data may be the numerical activity of the olfactory function or the numerical tissue activity of the prefrontal lobe. However, to provide a result with enhanced visibility to the examiner, there may be implemented a plurality of light receiving units. When a plurality of light receiving units 420a to 420h are included, an image may be created based on the activities individually obtained from the light receiving units or the obtained activities may be imaged. If the reflected light amount information is transferred from the plurality of light receiving units 420a to 420h to the diagnosis device 130, the diagnosis device 130 may generate and output the olfactory function activity or prefrontal lobe tissue activity, as an image or image data. Therefore, not only the examiner testing the dementia suspected patient but also the dementia suspected patient or his/her guardian may clearly recognize the result. Further, as the light receiving units 420a to 420h are disposed in different positions, it is possible to receive reflected light reflected in various directions.

The light receiving units 420a to 420h are disposed apart from the light sources 410 and 415 by a preset distance D. The distance between the light source and the light receiving unit is proportional to the degree of transmission of the light irradiated by the light source. Since the light irradiated from the light sources 410 and 415 disposed on the forehead of the dementia suspected patient needs to be transmitted up to, at least, the prefrontal lobe of the dementia suspected patient, the light receiving units 420a to 420h need to be disposed apart from the light sources 410 and 415 at, at least, the preset distance D. Here, the preset distance D may be twice the normal distance between the forehead and the prefrontal lobe. As such, since the light receiving units 420a to 420h need to be spaced apart from the light sources 410 and 415 by the predetermined distance, the light receiving units 420a to 420h may be arranged in a circle around the light sources 410 and 415 to minimize the overall volume of the dementia test device 120. Although FIG. 4 illustrates that eight light receiving units are provided, the present invention is not limited thereto, and the number of the light receiving units is not limited as long as a plurality of light receiving units are provided.

FIG. 5 is a view illustrating a configuration of a dementia test device according to a second embodiment of the present invention.

Referring to FIG. 5, the dementia test device 120 according to the second embodiment of the present invention includes light sources 510 and 515 and light receiving units 520, 522, 524 and 526. Unlike the dementia test device 120 according to the first embodiment, the dementia test device 120 according to the second embodiment may be included in the dementia diagnosis system 100 according to the first embodiment of the present invention.

The light sources 510 and 515 perform the same operation as the light sources 410 and 415.

The light receiving units 520 to 526 also perform the same operation as the light receiving units 420 to 426.

However, the light receiving units 520 to 526 are disposed apart from the light sources 510 and 515 by a preset distance as shown in FIG. 5(a) or are disposed apart from the light sources 510 and 515 by a preset distance or more as shown in FIG. 5(b). Here, the preset distance may be twice the normal distance between the forehead and the prefrontal lobe.

Further, the light receiving units 520 to 526 each are disposed to face both the left brain and the right brain. Rather than being randomly disposed around the forehead (a position, a predetermined distance away from the light sources), the light receiving units 520 to 526 are arranged so that a predetermined number of light receiving units face the left brain while the other light receiving units face the right brain. The light receiving units 520 to 526 may be arranged in such a way to be able to receive the light reflected from both the left brain and the right brain. Based on the data received by the light receiving units 520 to 526, the dementia diagnosis device 130 may not only derive tissue activity, such as metabolic activity or oxygen saturation, but also identify the connectivity between the left brain and the right brain, thus ensuring more accuracy in dementia diagnosis.

FIG. 6 is a view illustrating a configuration of a dementia diagnosis device according to the first embodiment of the present invention.

Referring to FIG. 6, according to an embodiment of the present invention, a dementia diagnosis device 130 includes a power source 601, a controller 620, and a display unit 630.

The power source 610 supplies power to each component in the dementia diagnosis device 130 and the dementia test device 120 so that each component operates.

The controller 620 controls the operation of the dementia test device 120 and analyzes and outputs the olfactory function activity or tissue activity of the prefrontal lobe based on the data received from the dementia test device 120.

The controller 620 controls the operation of the light sources in the dementia test device 120. A plurality of light sources 210 and 215 exist and each emits light of a different wavelength band. However, the light receiving units merely detect whether light is received and the amount of received light but may not detect what wavelength of light is received. Accordingly, the controller 620 may not recognize what wavelength of light the reflected light is about although receiving the reflected light amount from the light receiving units 220 to 226. To address this issue, the controller 620 controls the light irradiation timings of the light sources irradiating light of different wavelength bands. The controller 620 may control the light sources to alternately irradiate light or may control the light sources so that a light source for a specific wavelength band irradiates light during a predetermined time and another light source for another wavelength band irradiates light during the remaining time. As such, by controlling the operation of the light sources, the controller 620 may recognize which wavelength band the reflected light is about, even when each light source irradiates light of a different wavelength band.

The controller 620 controls the operations of the light receiving units 220 to 226 in the dementia test device 120. The light receiving unit has a different sensitivity to incident light depending on the bias voltage applied thereto. When the light receiving unit receives the reflected light as the light source operates, the controller 620 varies the magnitude of the bias voltage applied to some or all of the light receiving units 220 to 226. While the light receiving unit receives the reflected light, the controller 620 may alternately apply a lower magnitude of bias voltage and a higher magnitude of bias voltage to all of the light receiving units and may apply a lower magnitude of bias voltage to some of the light receiving units (in each of the light receiving units arranged to face the left brain and the right brain) and a higher magnitude of bias voltage to the other light receiving units (in each of the light receiving units arranged to face the left and right brains). As the controller 620 controls the bias voltage in this way, such an effect may be presented as if the dementia test device 120 includes light receiving units having different sensitivities. When the result values (tissue activity) are derived by the light receiving units having different sensitivities, each result value corresponds to a different item (category) used to determine the possibility of dementia. Unlike artificially increasing the amount of data to be stored, this may provide such an effect as if different types of tests are performed multiple times, which may increase the accuracy of dementia determination. Accordingly, the control of the bias voltage to the light receiving units by the controller 620 may produce such an effect as if the possibility of dementia is determined with more items even in a single test, thereby enhancing the accuracy of the determination of the possibility of dementia.

The controller 620 analyzes the olfactory function activity or the tissue activity of the prefrontal lobe based on the reflected light amount information received from the light receiving units. As described above, since the amount of reflected light is inversely proportional to the amount of hemoglobin present in the corresponding area, it is possible to figure out the amount of hemoglobin included in the tissue based on the amount of reflected light and to grasp the activity of the tissue based on the amount of hemoglobin. The controller 620 may analyze the tissue activity of the prefrontal lobe based on the information for the amount of reflected light received from the light receiving units to thereby grasp the activity of the olfactory function of the dementia suspected patient. Since the plurality of light receiving units receive the reflected light and each of the light receiving units transfers the reflected light amount information, the controller 620 may produce an image or video of the analyzed activity of the tissue. The controller 620 controls the display unit 630 to output the image or video of the tissue activity.

The display unit 640 outputs the produced image or video of the tissue activity.

As such, since the dementia diagnosis device 130 analyzes the activity of the prefrontal lobe tissue of the dementia suspected patient for the scent discharged from a preset distance away, the examiner may simply place the test container 110 on the olfactory organ of the dementia suspected patient so that analysis may be done as how much the tissue has been activated. Accordingly, with the dementia diagnosis system according to the first embodiment of the present invention, it is possible to derive an analysis result only with one test, and it is also possible to derive the same result even when the test environment or the examiner is different. It is possible to address the issues with the conventional methods, e.g., those providing a questionnaire with many questions and obtaining answers from dementia suspected patients, which are time-consuming and give inaccurate results due to relying on the cognitive ability of the dementia suspected patient (for the questions). Further, since accurate dementia diagnosis may be performed even from a single test, it is possible to ensure the convenience for the dementia patient and the accuracy of the diagnosis.

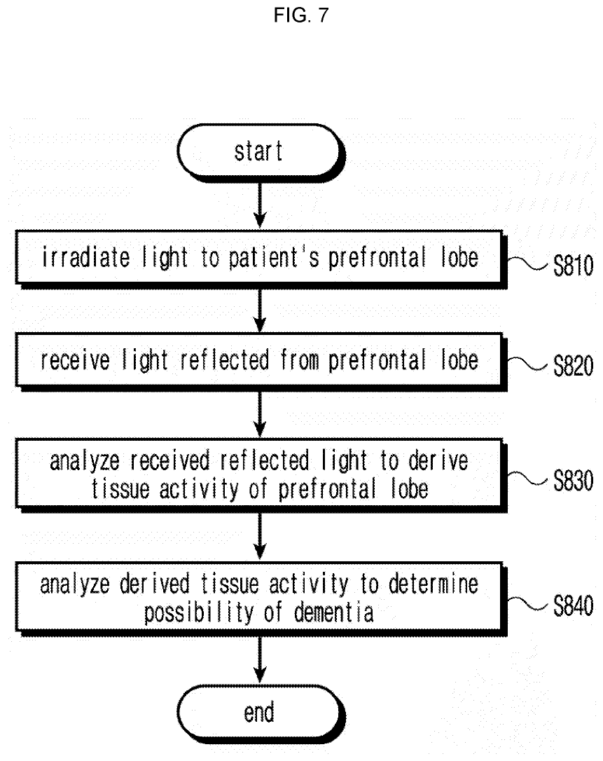

FIG. 7 is a flowchart illustrating a method for diagnosing a possibility of dementia for a patient by a dementia diagnosis system according to the first embodiment of the present invention.

The dementia test device 120 irradiates light to the patient's prefrontal lobe (S810).

The dementia test device 120 receives the light reflected from the prefrontal lobe after being irradiated (S820).

The dementia diagnosis device 130 analyzes the received reflected light to derive the tissue activity of the prefrontal lobe (S830).

The dementia diagnosis device 130 analyzes the derived tissue activity to determine the possibility of dementia for the patient (S840). The dementia diagnosis device 130 may output a determination result.

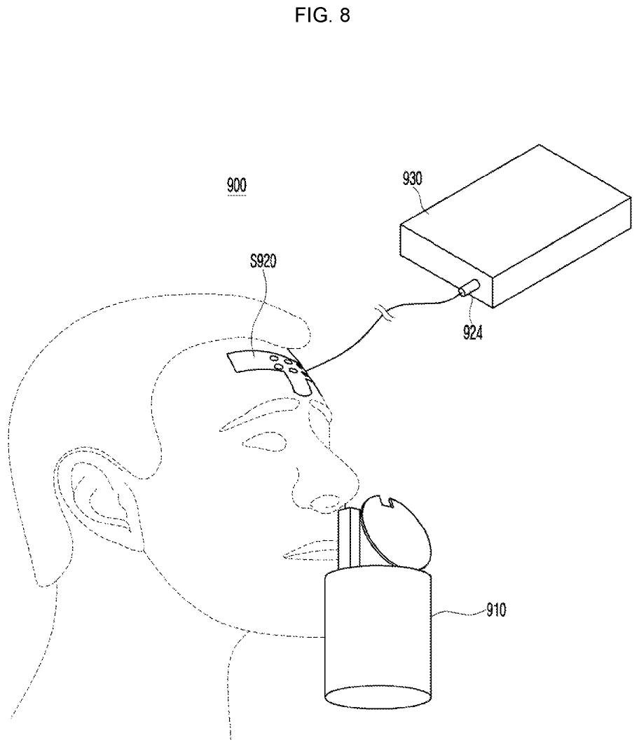

FIG. 8 is a view illustrating a configuration of a dementia diagnosis system according to a third embodiment of the present invention.

Referring to FIG. 8, the dementia diagnosis system 900 according to the third embodiment of the present invention includes a test container 910, a dementia test device 920, and a dementia diagnosis device 930.

The test container 910 performs the same operation as the test container 110 in the dementia diagnosis system 100 according to the first embodiment of the present invention.

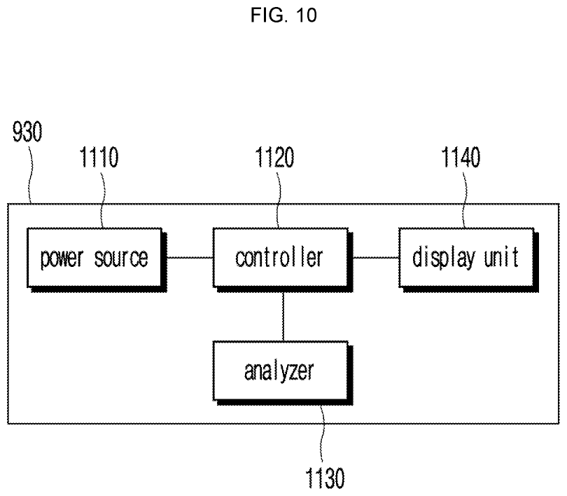

The dementia test device 920 is attached to a preset portion of a dementia suspected patient and examines the concentration of hemoglobin for activating the olfactory function or the function of the prefrontal lobe. Since the test container 910 is provided to provide a scent to the dementia suspected patient, the olfactory function or the function of the prefrontal lobe of the dementia suspected patient is in an activated state. The dementia test device 920 is attached to a preset portion of the dementia suspected patient and measures the concentration of hemoglobin moved to the prefrontal lobe to activate the function of the prefrontal lobe or olfactory function of the dementia suspected patient or the concentration of hemoglobin already moved to the prefrontal lobe. The dementia test device 920 examines whether a relatively small amount or excessive amount of oxygen (hemoglobin) is provided to the prefrontal lobe by the olfactory stimulus due to intensive degeneration of prefrontal lobe (degeneration of olfactory function thereby) shown to dementia patients. The dementia test device 920 is attached near the forehead of the dementia suspected patient for the above-mentioned examination. The dementia test device 920 is attached to the forehead of the dementia suspected patient and then examines the concentration of hemoglobin in the prefrontal lobe of the dementia suspected patient. The dementia test device 920 is described below in greater detail with reference to FIG. 10.

The dementia test device 920 includes a connector 924, connected with the dementia diagnosis device 930, at one end thereof.

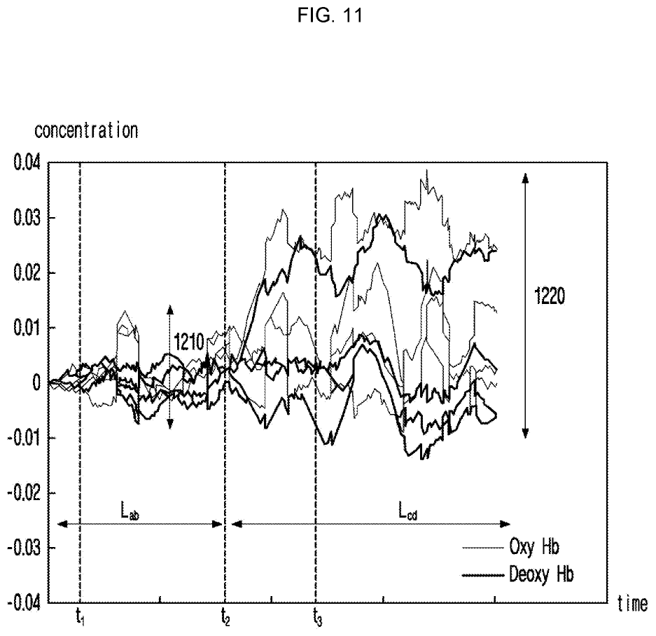

The dementia diagnosis device 930 analyzes the result of examination by the dementia test device 920 to derive the concentration of hemoglobin (concentration of deoxy hemoglobin), which has moved to the prefrontal lobe of the dementia suspected patient, or the concentration of hemoglobin (concentration of oxy hemoglobin), which is moved to the prefrontal lobe of the dementia suspected patient, and analyzes the derived results to diagnose the possibility of dementia. The dementia diagnosis device 930 analyzes the test results of the dementia test device 920, derives how much hemoglobin moves or has moved to the vicinity of the patient's prefrontal lobe to activate the olfactory function of the dementia suspected patient by smelling the scent from the test container 910. In other words, the dementia diagnosis device 930 derives the concentration of hemoglobin that moves or has already moved to the vicinity of the prefrontal lobe based on the test results. The dementia diagnosis device 930 analyzes the dementia possibility index from the derived result and diagnoses the possibility of dementia for the dementia suspected patient. The dementia diagnosis device 930 is described below in greater detail with reference to FIG. 11.

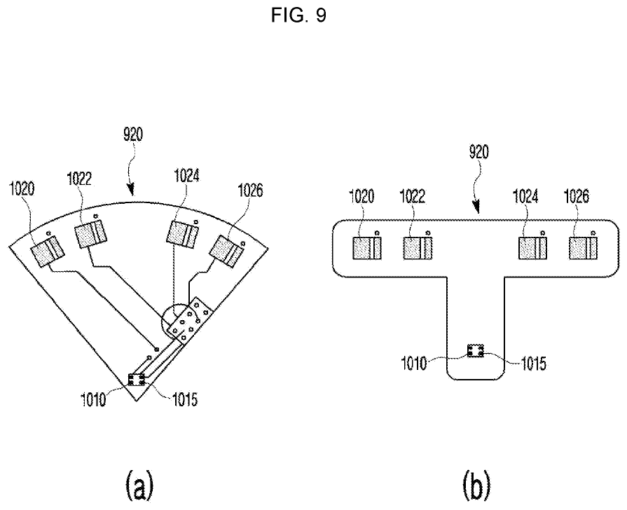

FIG. 9 is a view illustrating a configuration of a dementia test device according to the third embodiment of the present invention.

Referring to FIG. 9, the dementia test device 920 according to the third embodiment of the present invention includes light sources 1010 and 1015 and light receiving units 1020, 1022, 1024 and 1026.

The light sources 1010 and 1015 perform similar operation to the light sources 410 and 415.

The light sources 1010 and 1015 irradiate light in a near-infrared wavelength band. Light in the near-infrared wavelength band has the property of being absorbed by hemoglobin. If the prefrontal lobe contains a lot of hemoglobin, the absorption of near-infrared light increases and the light reflection from the prefrontal lobe decreases. Since hemoglobin is an oxygen carrier unless there is a special circumstance (e.g., carbon monoxide poisoning), hemoglobin needs to be moved to a specific tissue (the olfactory organ according to embodiments of the present invention) and its related tissues (the prefrontal lobe according to embodiments of the present invention) to activate the specific tissue. Accordingly, the light sources 1010 and 1015 irradiate near-infrared light into the patient's body to examine the concentration of hemoglobin moving to the prefrontal lobe of the dementia suspected patient due to activation of the olfactory function of the dementia suspected patient.

The light sources 1010 and 1015 irradiate near-infrared light having different wavelengths. One of the light sources 1010 and 1015 irradiates light of a 730 nm band, and the other irradiates light of an 850 nm band. There are two types of hemoglobin that migrate to the prefrontal lobe: oxy hemoglobin (Oxy Hb) to provide oxygen to the prefrontal lobe and deoxy hemoglobin (Deoxy Hb) that has already provided oxygen to the prefrontal lobe. At least two light sources 1010 and 1015 irradiate near-infrared light of different wavelengths so that the dementia diagnosis device 930 may derive the concentration of each type of hemoglobin.

The light receiving units 1020 to 1026 are disposed apart from the plurality of light sources 1010 and 1015 by a predetermined distance, receiving the light reflected from the prefrontal lobe.

The light receiving units 420a to 420h receive the light reflected from the prefrontal lobe a preset number of times per hour. The light receiving units 420a to 420h receive the light reflected, without being absorbed by hemoglobin, among the near-infrared light irradiated to the prefrontal lobe of the dementia suspected patient. Each of the light receiving units 420a to 420h transfers the sensed reflected light amount information to the dementia diagnosis device 930 so that the dementia diagnosis device 930 may derive the concentration of hemoglobin based on based on the amount of the reflected light. In this case, each of the light receiving units 420a to 420h may receive light a preset number of times per predetermined time. For example, each of the light receiving units 420a to 420h may receive reflected light 20 times per second.

Further, the light receiving units 1020 to 1026 are disposed in the same or a similar manner to the light receiving units 520 to 526.

FIG. 10 is a view illustrating a configuration of a dementia diagnosis device according to the third embodiment of the present invention.

Referring to FIG. 10, according to the third embodiment of the present invention, a dementia diagnosis device 930 includes a power source 1110, a controller 1120, an analyzer 1130, and a display unit 1140.

The power source 1110 and the controller 1120 perform the same operations as the power source 610 and the controller 620 in the dementia diagnosis device 130.

The controller 1120 controls the operations of the light receiving units 1020 to 1026 in the dementia test device 920. The light receiving unit has a different sensitivity to incident light depending on the bias voltage applied thereto. When the light receiving unit receives the reflected light as the light source operates, the controller 1120 varies the magnitude of the bias voltage applied to some or all of the light receiving units 1020 to 1026. While the light receiving unit receives the reflected light, the controller 1120 may alternately apply a lower magnitude of bias voltage and a higher magnitude of bias voltage to all of the light receiving units and may apply a lower magnitude of bias voltage to some of the light receiving units (in each of the light receiving units arranged to face the left brain and the right brain) and a higher magnitude of bias voltage to the other light receiving units (in each of the light receiving units arranged to face the left and right brains). As the controller 1120 controls the bias voltage in this way, such an effect may be presented as if the dementia test device 920 includes light receiving units having different sensitivities. When the result values (tissue activity) are derived by the light receiving units having different sensitivities, each result value corresponds to a different item (category) used to determine the possibility of dementia. Unlike artificially increasing the amount of data to be stored, this may provide such an effect as if different types of tests are performed multiple times, which may increase the accuracy of dementia determination. Accordingly, the control of the bias voltage to the light receiving units by the controller 1120 may produce such an effect as if the possibility of dementia is determined with more items even in a single test, thereby enhancing the accuracy of the determination of the possibility of dementia.

The analyzer 1130 derives the hemoglobin concentrations before and after the scent is provided to the dementia suspected patient, based on the sensing values sensed by each of the light receiving units 1020 to 1026 and diagnoses the possibility of dementia from the derived results.

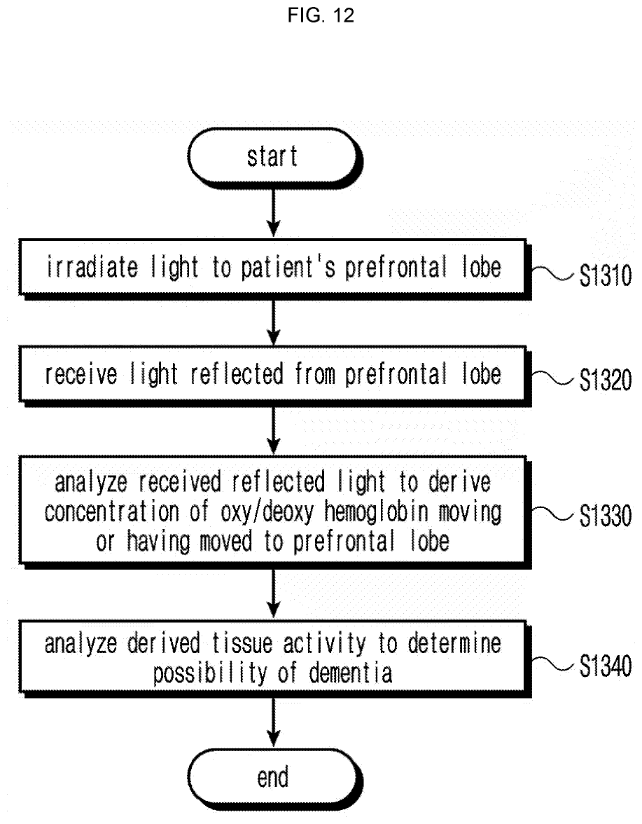

The analyzer 1130 derives the hemoglobin concentration at each time before and after the scent is provided to the dementia suspected patient, based on the sensing values sensed by each of the light receiving units 1020 to 1026. The analyzer 1130 derives the concentrations of oxy hemoglobin and deoxy hemoglobin from the sensing values sensed by each of the light receiving units 1020 to 1026 using various equations, such as the Beer-Lambert law. In order for the analyzer 1130 to derive both the concentrations of oxy hemoglobin and deoxy hemoglobin, the dementia test device 920 performs the test using at least two light sources irradiating light of different wavelengths. However, since the dementia test device 920 includes basic light sources and light-receiving units, rather than expensive equipment, such as MRI or precise devices, such as a laser diode, the absolute values of the derived hemoglobin concentrations may be less accurate. Accordingly, in deriving the concentrations of oxy hemoglobin and deoxy hemoglobin at each time based on the sensed values, the analyzer 1130 derives relative values of the concentrations. A method in which the analyzer 1130 derives the relative values of the concentrations is described with reference to FIG. 12.

FIG. 11 is a graph illustrating the hemoglobin concentration of a patient analyzed by a dementia diagnosis device according to the third embodiment of the present invention.

The graph depicts the concentration of oxy hemoglobin (Oxy Hb) and the concentration of deoxy hemoglobin (Deoxy Hb) calculated by the sensing values sensed from each light receiving unit. Here, the concentration of oxy hemoglobin before the time t₂ when the scent is provided is defined as a, the concentration of deoxy hemoglobin before the time t₂ when the scent is provided is defined as b, the concentration of oxy hemoglobin after the time t₂ when the scent is provided is defined as c, and the concentration of deoxy hemoglobin after the time t₂ when the scent is provided is defined as d. Further, a first preset period L_{ab} up to the time t₂ when the scent is provided to the dementia suspected patient and a second preset period L_{cd} starting from the time when the scent is provided are defined.

First, the analyzer 1130 derives concentrations of oxy hemoglobin and deoxy hemoglobin corresponding to reference values for deriving the relative values of hemoglobin concentrations at each time. The reference value is an average value of the hemoglobin concentration from when the dementia test device 920 is mounted to a preset time t₁ before the scent is provided to the dementia suspected patient. The analyzer 1130 calculates the reference values and may calculate the relative values of the hemoglobin concentrations at each time after the preset time t₁ using the reference values.

Thereafter, the analyzer 1130 derives the relative value of the hemoglobin concentration at each time in the first preset period L_{ab}. The analyzer 1130 derives a relative value with respect to the reference value from the absolute value of the hemoglobin concentration (derived from the sensed value) at each time based on the above-described reference value. Referring to the graph of FIG. 12, it is shown that variations 1210 in the concentration of oxy hemoglobin and deoxy hemoglobin are relatively small in the period L_{ab} during which the scent is not provided.

Here, the time t₂ at which the scent is provided may be set to a value ranging from 30 seconds to 50 seconds. If the scent is provided before 30 seconds, it may be difficult to calculate the reference value which is necessary for calculating the relative value of the hemoglobin concentration. On the other hand, if the scent is provided after 50 seconds, the dementia suspected patient may think a different thing so that the sensing value sensed by the light receiving unit may be varied. Since the sensing value sensed by the light receiving unit is the amount of light reflected from the prefrontal lobe, the value does not necessarily change only when the olfactory function is activated. Even when the olfactory function is not activated, if the dementia suspected patient has various thoughts, the prefrontal lobe may also be activated, causing a reduction in the accuracy of data. Thus, the time t₂ at which the scent is provided may be set to a value ranging from 30 seconds to 50 seconds.

Likewise, the analyzer 1130 derives the relative value of the hemoglobin concentration at each time in the second preset period L_{cd}. In the second preset section L_{cd}, the scent is provided only from time t₂ to time t₃ and is not provided in the other periods (after t₃). The period t₂ to t₃ during which the scent is provided may be set to 10 seconds to 30 seconds. If the scent is provided for less than 10 seconds, there is a possibility that the activation of the olfactory sense will not be sufficient. On the other hand, if the scent is provided for longer than 30 seconds, the olfactory sense is used to the scent, and when the patient thinks other things or by other stimuli applied to the patient, stimulation may occur in the prefrontal lobe, so that the source of the stimulation in the prefrontal lobe becomes incorrect. Further, since reaction to the scent is immediate for some patients while reaction occurs a predetermined time later for other patients, the scent is provided as long as 10 seconds to 30 seconds. The analyzer 1130 derives a relative value of the hemoglobin concentration in the period during which the scent is provided.

Thereafter, while no scent is provided for 30 seconds to 50 seconds after the scent has been provided (t₃), the analyzer 1130 derives the relative value of the hemoglobin concentration. Since the second preset period L_{cd} is after the scent has been provided, the variation 1220 in the relative value of the hemoglobin concentration relatively increases.

The analyzer 1130 analyzes the dementia possibility index based on the so-derived relative value of the hemoglobin concentration and diagnoses the possibility of dementia for the dementia suspected patient. Since numerous relative values of oxy hemoglobin concentrations and relative values of deoxy hemoglobin concentrations are calculated at each time, the analyzer 1130 calculates the average value of the hemoglobin concentrations in each period L_{ab} and L_{cd}. The analyzer 1130 calculates the average of the hemoglobin concentrations in each period by calculating each of is/L_{ab}, Σb/L_{ab}, Σd/L_{cd} and Σ/L_{cd}. Thereafter, the analyzer 1130 analyzes the dementia possibility index. The dementia possibility index is calculated as a ratio between the average value of the concentration of oxy-deoxy hemoglobin before the scent is provided and the average value of the concentration of oxy-deoxy hemoglobin after the scent is provided. In other words, the dementia probability index is as follows.

$$\begin{matrix}dementia possibility index = \frac{\begin{matrix}average oxy ‐ deoxy hemoglobin \\ concentration before providing scent\end{matrix}}{\begin{matrix}average oxy ‐ deoxy hemoglobin \\ concentration after providing scent\end{matrix}} \\ = \frac{\frac{\sum \vert α \vert + \sum \vert b \vert}{{L}_{ab}}}{\frac{\sum \vert c \vert + \sum \vert d \vert}{{L}_{c d}}}\end{matrix}$$

The dementia possibility index is calculated as in the above-mentioned formula and represents the variation rate of the hemoglobin concentration between before and after the scent is provided. The analyzer 1130 determines whether the concentrations before and after the scent is provided from the index have changed by more than a preset ratio. Here, the preset ratio may be 15%. In other words, if the concentration changes by 15% or more (the index is 1.15 or more or 0.85 or less), the analyzer 1130 diagnoses the patient as likely to have dementia. A large change in the concentration of hemoglobin means that a small amount of hemoglobin has been transferred to the prefrontal lobe or that an excessive amount of hemoglobin has been transferred to the prefrontal lobe. If a small amount of hemoglobin is transported to the prefrontal lobe, it means that the olfactory function or tissue activity of the prefrontal lobe decreases. Similarly, the transfer of an excessive amount of hemoglobin to the prefrontal lobe means that the prefrontal lobe requires a greater amount of hemoglobin to process the same level of olfactory stimulus, which also suggests that the olfactory function or tissue activity of the prefrontal lobe decreases. Accordingly, the analyzer 1130 diagnoses the possibility of dementia for the patient based on the index analyzed from the average value of the derived hemoglobin concentrations (relative values). However, the preset ratio is not necessarily limited to 15% and may be changed in some cases.

Referring back to FIG. 10, the display unit 1140 outputs the results of determination of the analyzer 1130.

As such, since the dementia diagnosis device 930 analyzes the possibility of dementia for the dementia suspected patient for the scent discharged from a preset distance away, the examiner may simply place the test container 110 on the olfactory organ of the dementia suspected patient so that analysis may be done as how much the tissue has been activated. Accordingly, with the dementia diagnosis system according to an embodiment of the present invention, it is possible to derive an analysis result only with one test, and it is also possible to derive the same result even when the test environment or the examiner is different. It is possible to address the issues with the conventional methods, e.g., those providing a questionnaire with many questions and obtaining answers from dementia suspected patients, which are time-consuming and give inaccurate results due to relying on the cognitive ability of the dementia suspected patient (for the questions). Further, since accurate dementia diagnosis may be performed even from a single test, it is possible to ensure the convenience for the dementia patient and the accuracy of the diagnosis.

FIG. 12 is a flowchart illustrating a method for diagnosing a possibility of dementia for a patient by a dementia diagnosis system according to the third embodiment of the present invention.

The dementia test device 920 irradiates light to the patient's prefrontal lobe (S1310).

The dementia test device 920 receives the light reflected from the prefrontal lobe after being irradiated (S1320).

The dementia diagnosis device 930 analyzes the received reflected light and derives the concentration of oxy/deoxy hemoglobin that moves to the prefrontal lobe or has already moved to the prefrontal lobe (S1330). The dementia diagnosis device 930 analyzes the received reflected light, deriving the concentrations of oxy-deoxy hemoglobin that moves or has moved to the prefrontal lobe before the scent is provided and the concentrations of oxy/deoxy hemoglobin that moves or has moved to the prefrontal lobe after the scent is provided.

The dementia diagnosis device 930 analyzes the derived concentrations of oxy-deoxy hemoglobin and determines the possibility of dementia for the patient (S1340). The dementia diagnosis device 130 calculates a dementia possibility index based on the derived concentrations of oxy/deoxy hemoglobin before and after the scent is provided and determines the possibility of dementia for the dementia suspected patient therefrom.

Although FIGS. 8 and 13 illustrate that the steps are sequentially performed, this merely provides an embodiment of the disclosure. It would readily be appreciated by a skilled artisan that the steps of FIGS. 8 and 13 are not limited to the order shown but may rather be performed in a different order, one or more of the steps may simultaneously be performed, or other various modifications or changes may be made thereto without departing from the scope of the disclosure

The steps or processes described above in connection with FIGS. 8 and 13 may be implemented as computer-readable code in a recording medium. The computer-readable recording medium includes all types of recording devices storing data readable by a computer system. The computer-readable recording medium includes a storage medium, such as a magnetic storage medium (e.g., a ROM, a floppy disk, or a hard disk) or an optical reading medium (e.g., a CD-ROM or a DVD). Further, the computer-readable recording medium may be distributed to computer systems connected via a network, and computer-readable codes may be stored and executed in a distributed manner.

The above-described embodiments are merely examples, and it will be appreciated by one of ordinary skill in the art various changes may be made thereto without departing from the scope of the present invention. Accordingly, the embodiments set forth herein are provided for illustrative purposes, but not to limit the scope of the present invention, and should be appreciated that the scope of the present invention is not limited by the embodiments. The scope of the present invention should be construed by the following claims, and all technical spirits within equivalents thereof should be interpreted to belong to the scope of the present invention.

### CROSS-REFERENCE TO RELATED APPLICATION

The instant patent application claims priority under 35 U.S.C. 119(a) to Korean Patent Application No. 10-2019-0120144, filed on Sep. 27, 2019, in the Korean Intellectual Property Office, the disclosure of which is herein incorporated by reference in its entirety. The present patent application claims priority to other applications to be filed in other countries, the disclosures of which are also incorporated by reference herein in their entireties.

1. A dementia diagnosis device examining a possibility of dementia for a dementia suspected patient by analyzing an amount of light reflected from the dementia suspected patient's prefrontal lobe in a situation where a scent for examination is provided to the dementia suspected patient, the dementia diagnosis device comprising:
an analyzer deriving a concentration of hemoglobin moving or having moved to the patient's prefrontal lobe before the scent is provided to the patient's olfactory organ and a concentration of hemoglobin moving or having moved to the patient's prefrontal lobe after the scent is provided to the patient's olfactory organ from the amount of the reflected light and analyzing a dementia possibility index based on the derived concentrations to diagnose the possibility of dementia for the dementia suspected patient.

2. The dementia diagnosis device of claim 1, wherein the analyzer derives each of a concentration of oxy hemoglobin (Oxy Hb) and a concentration of deoxy hemoglobin (Deoxy Hb) in deriving the concentrations of hemoglobin.

3. The dementia diagnosis device of claim 2, wherein the concentration of hemoglobin is a relative value with respect to a concentration of hemoglobin measured at each time and a preset reference value.

4. The dementia diagnosis device of claim 1, wherein the analyzer determines whether the concentrations of hemoglobin before and after the scent is provided are varied by a preset ratio or more from the dementia possibility index and diagnoses the possibility of dementia for the dementia suspected patient according to a result of the determination.

5. A method for examining, by a dementia diagnosis device, a possibility of dementia for a dementia suspected patient by analyzing an amount of light reflected from the dementia suspected patient's prefrontal lobe in a situation where a scent for examination is provided to the dementia suspected patient, the method comprising:
deriving a concentration of hemoglobin moving or having moved to the patient's prefrontal lobe before the scent is provided to the patient's olfactory organ and a concentration of hemoglobin moving or having moved to the patient's prefrontal lobe after the scent is provided to the patient's olfactory organ from the amount of the reflected light; and
analyzing a dementia possibility index based on the derived concentrations to diagnose the possibility of dementia for the dementia suspected patient.

6. The method of claim 5, wherein the deriving derives each of a concentration of oxy hemoglobin (Oxy Hb) and a concentration of deoxy hemoglobin (Deoxy Hb) in deriving the concentrations of hemoglobin.

7. A dementia diagnosis system examining a possibility of dementia for a patient, the dementia diagnosis system comprising:
a test container providing a scent to the patient's olfactory organ;
a dementia test device irradiating light of a preset wavelength band to the patient's prefrontal lobe and receiving light reflected from the patient's prefrontal lobe; and
a dementia diagnosis device analyzing information for the reflected light received by the dementia test device to derive a concentration of hemoglobin moving or having moved to the patient's prefrontal lobe before the scent is provided to the patient's olfactory organ and a concentration of hemoglobin moving or having moved to the patient's prefrontal lobe after the scent is provided to the patient's olfactory organ from the amount of the reflected light and analyzing a dementia possibility index based on the derived concentrations to diagnose the possibility of dementia for the dementia suspected patient.
